(19) United States
(12) Patent Application Publication
(10) Pub. No.: US 2012/0130486 A1
(43) Pub. Date: May 24, 2012

(54) MULTIFOCAL OPHTHALMIC LENS DESIGNS USING WAVEFRONT INTERACTION

(75) Inventor: Geunyoung YOON, Pittsford, NY (US)

(73) Assignee: University of Rochester, Rochester, NY (US)

(21) Appl. No.: 13/295,791

(22) Filed: Nov. 14, 2011

Related U.S. Application Data
(60) Provisional application No. 61/414,076, filed on Nov. 16, 2010.

(51) Int. Cl. A61F 2/16; G02C 7/02

(52) U.S. Cl. 623/6.11; 351/159.01

(57) ABSTRACT

An intraocular lens introduces higher order aberrations, e.g., spherical aberration (SA), for different sub-optical zone diameters. For example, a lens may have a central sub-optical zone which introduces a spherical aberration. Alternatively, a full optical zone design can be used combining defocus with a higher-order aberration.

### REFERENCE TO RELATED ART

The present application claims the benefit of U.S. Provisional Patent Application No. 61/414,076, filed Nov. 16, 2010, whose disclosure is hereby incorporated by reference in its entirety into the present disclosure.

### FIELD OF THE INVENTION

The present invention is directed to treatment of presbyopia and more particularly to such treatment using ophthalmic lenses or other refractive error correction methods with different optical zones.

### DESCRIPTION OF RELATED ART

The amplitude of human lens accommodation, the eye's ability to change power dynamically, is decreased with aging and at the age of 60, very limited accommodation is available. That age-related lack of accommodation is called “presbyopia,” and its effects are shown in FIG. 1.

Since that happens to everybody at some point in life, there is a huge demand for therapeutic tools. Those include spectacles (bifocal or progressive addition lens), multifocal contact lenses or intraocular lenses (IOL's), surgical alterations, injection of elastic polymer gel, etc. Multifocal contact lenses or IOL's which increase depth of focus are one of the popular options which have been used to overcome presbyopia. Those lenses, however, have to compromise retinal image quality in distant vision to enhance image quality of a near object.

These commercially available multifocal IOLs have very poor intermediate vision, although they provide reasonably acceptable quality for distance and near images. This is a significant limitation that the present invention seeks to overcome.

FIGS. 2A-2D show known IOL's for treatment of presbyopia, respectively, the ReSTOR® (diffractive), ReZoom® (refractive), Crystalens® (accommodating single optic) and Synchrony® (accommodating dual optic). FIG. 2E shows in greater detail the Bausch & Lomb Crystalens HD™; the central portion controls near focus, while the outer portion controls distant focus. FIG. 3 shows the effectiveness of such IOL's in extending depth of focus.

### SUMMARY OF THE INVENTION

It is therefore an object of the invention to enhance the depth of focus.

To achieve the above and other objects, the invention introduces wavefront interaction between defocus and higher order aberrations, e.g., spherical aberrations (SA) and other higher order SAs, for either full optical zone or different sub-optical zone diameters. For example, a lens may have a central sub-optical zone which introduces a SA or combination of multiple SAs.

The present invention provides a way to optimize through-focus performance in terms of overall image quality and depth of focus. The same idea can be used for both full and partial optical zones. Each of primary and higher order (secondary, tertiary, forth order and so on) spherical aberrations can be used. These aberrations can be combined to further improve the performance. The present invention can be extended to binocular wavefront manipulation e.g. modified monovision. The present invention can be implemented in any suitable way, e.g., with an intraocular or contact lens or any appropriate surgical technique.

### BRIEF DESCRIPTION OF THE DRAWINGS

A preferred embodiment of the present invention will be set forth in detail with reference to the drawings, in which:

FIG. 1 is a diagram that shows the effects of presbyopia;

FIGS. 2A-2E show conventional intraocular lenses used to treat presbyopia;

FIG. 3 is a series of images showing the effectiveness of commercially available intraocular lenses in extending depth of focus;

FIG. 4 is a chart that shows the effects of two types of SA (primary and secondary) on through focus image quality;

FIG. 5 is a graph showing the ability of SA's to optimize near image quality;

FIG. 6 is a graph showing that SA interacts linearly with defocus;

FIG. 7 is a graph and a series of images showing that primary SA optimizes intermediate viewing, while secondary SA optimizes near viewing;

FIG. 8 is series of plots of image quality tradeoffs;

FIG. 9 shows an intraocular lens according to the preferred embodiment; and

FIG. 10 is a series of images showing the effects of the intraocular lens of the preferred embodiment.

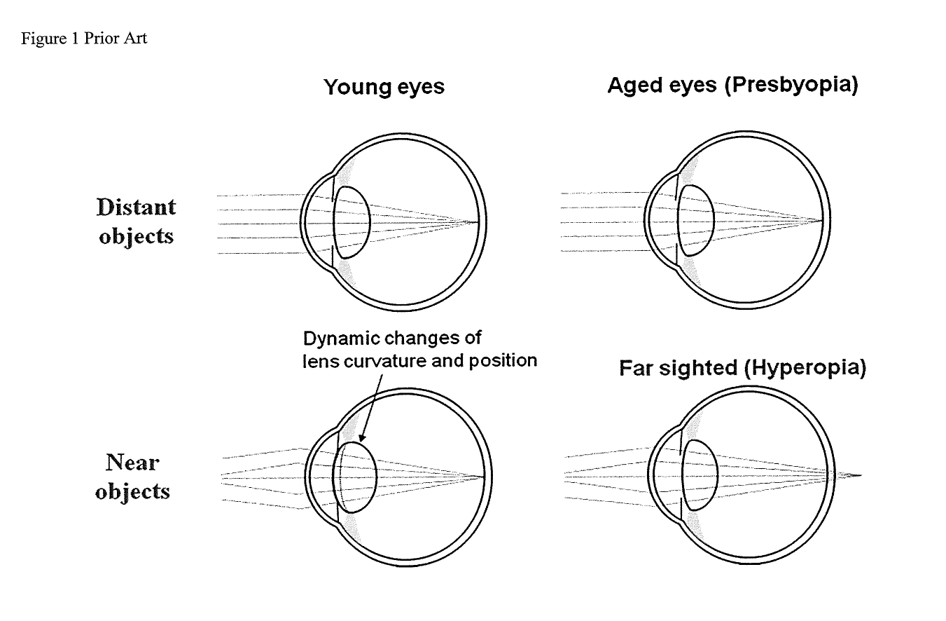

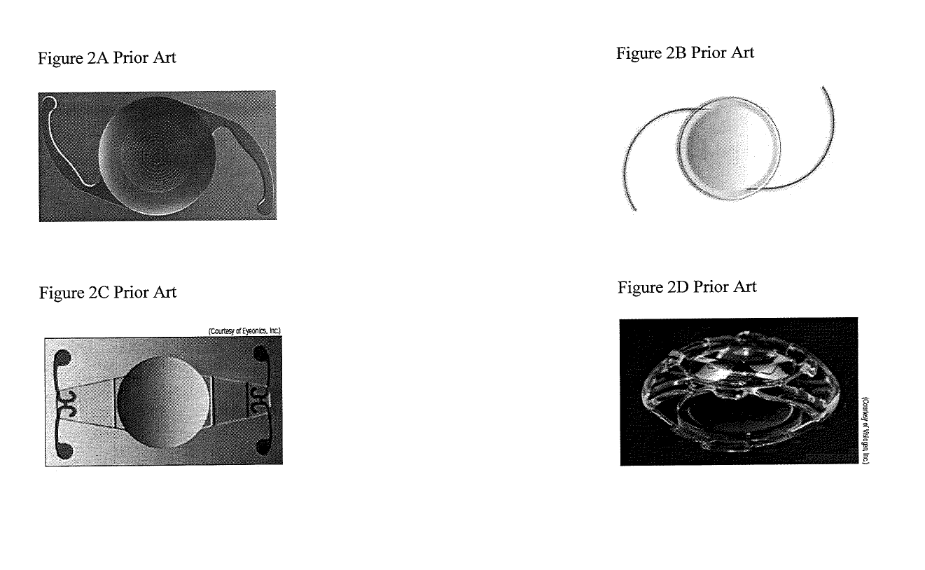

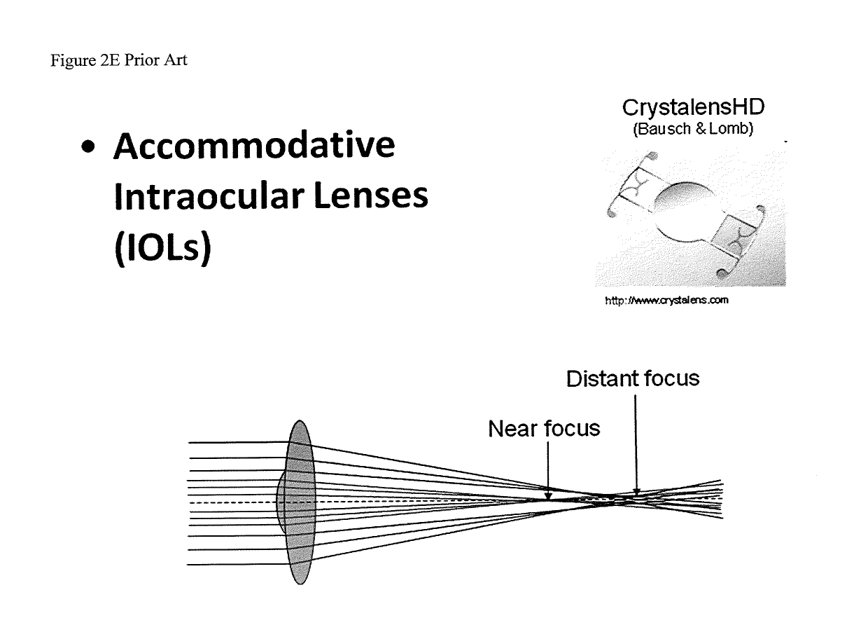

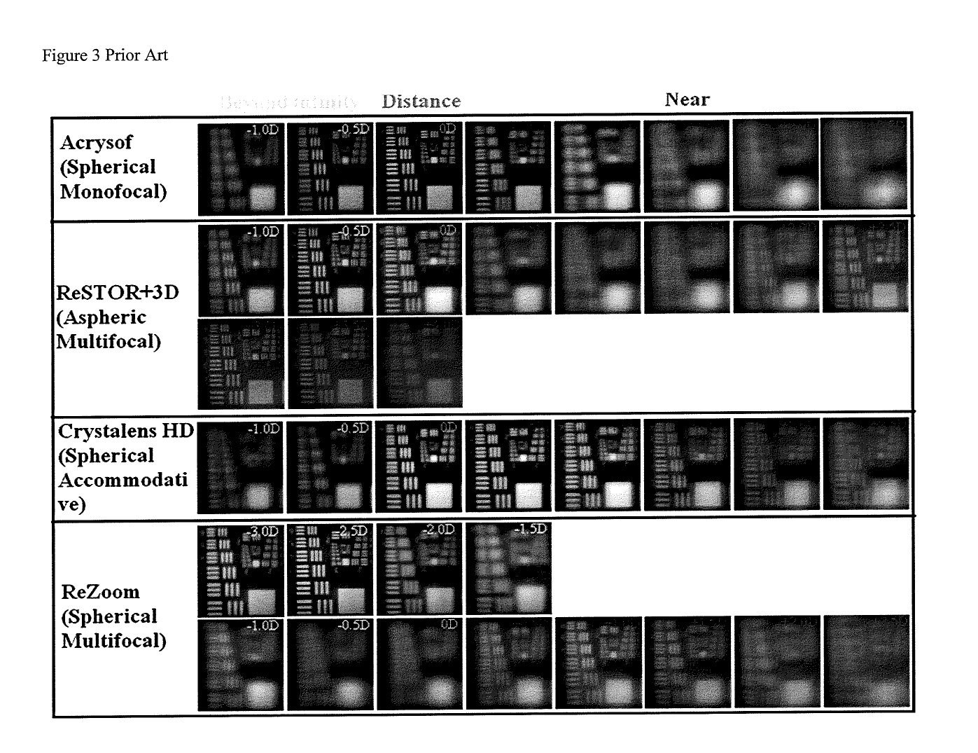

### DETAILED DESCRIPTION OF THE PREFERRED EMBODIMENT

A preferred embodiment of the present invention will be set forth in detail with reference to the drawings.

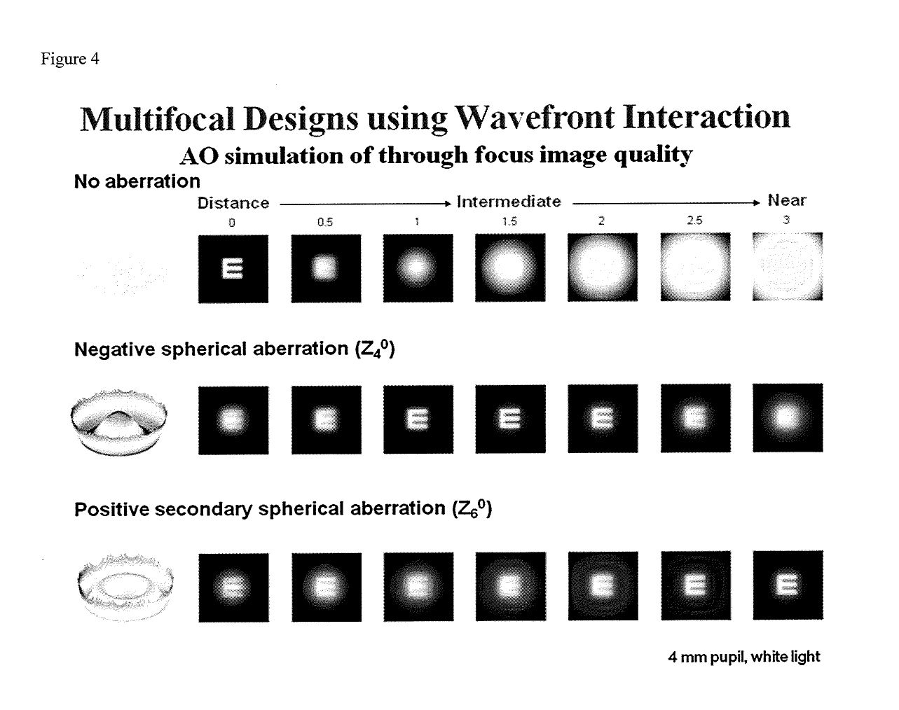

FIG. 4 shows an AO (adaptive optics) simulation of through focus image quality. The top row shows the case of no aberration. The middle row shows the case of negative primary SA (in terms of Zernike polynomials, Z₄⁰). The bottom row shows the case of positive secondary SA (Z₆⁰). As can be seen, both types of aberrations dramatically improve image quality for various distances and more interestingly, these two SAs have different characteristics in that primary SA optimizes intermediate image quality while secondary SA improves distance and near image quality This indicates that balancing these two (or more) SAs can enhance through-focus image quality.

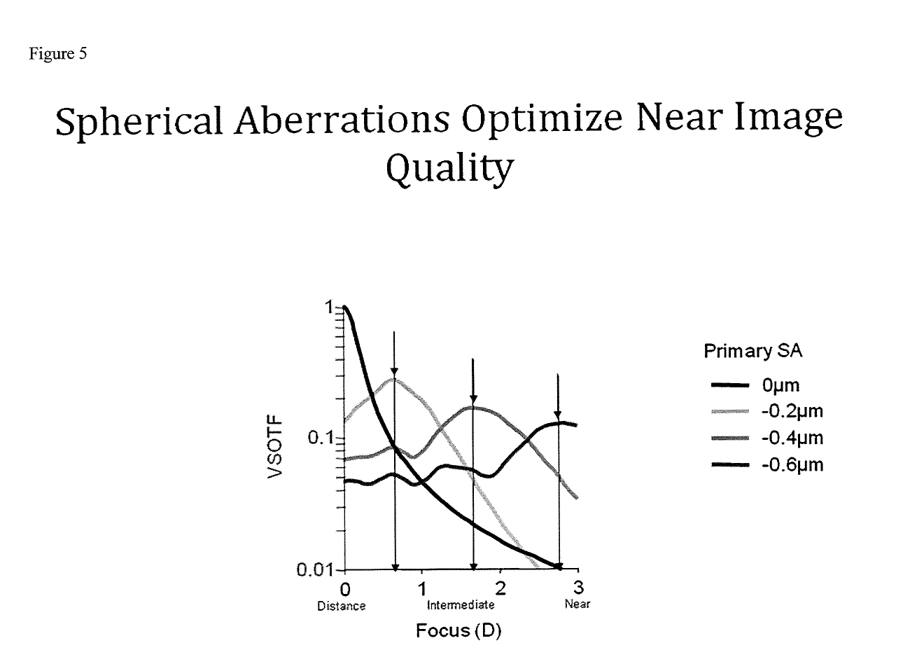

FIG. 5 shows a plot of the VSOTF (visual Strehl ratio based on the optical transfer function, an image quality metric) through a focus range of OD, representing distance vision or looking far away, to near, which is 3D or a comfortable reading distance of 33 cm. The intermediate zone is approximately 67 cm, where a computer monitor would be. That figure illustrates that for an unaberrated system, distance image quality is quite good, but in near viewing, the image quality degrades significantly. However, adding a small amount of primary SA causes a degradation in distance visual quality but an optimization of image quality at a nearer focus position. In addition, the depth of focus has been extended. Similarly, adding larger amounts of primary SA through focus results in an even greater degradation in the distance image quality, but with the optimizations at even nearer focus points, which further extends the depth of focus. This leads us to conclude that an optimized object distance where the image quality is peak depends on the magnitude of induced SA. It is also important to note that loss of image quality at different object distances increases with an increase in the magnitude of SA. The same trend can be seen in the case of secondary SAs.

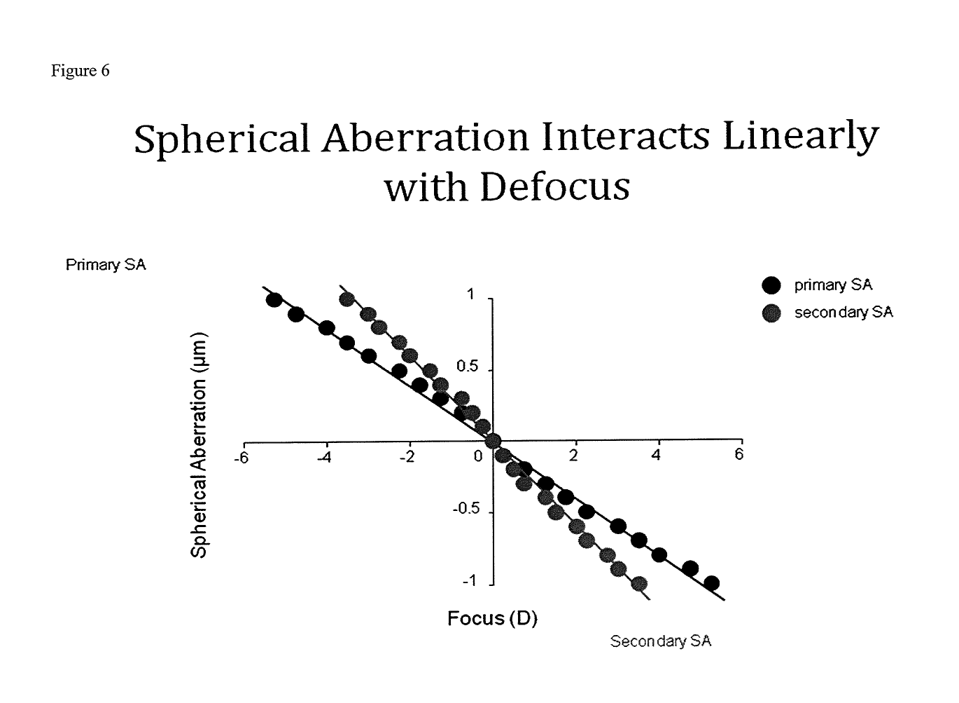

The graph shows a relationship between primary SA and focus position where the image quality has been optimized for near viewing. That relationship between focus position of optimized image quality and the SA shows that SA interacts linearly with defocus, as shown in FIG. 6. The less steep curve represents primary SA, which is proportional to

$$( \frac{{r}^{2}}{- 12 \sqrt{3}} )$$

times focus, while the steeper curve represents secondary SA, which is proportional to

$$( \frac{{r}^{2}}{- 8 \sqrt{3}} )$$

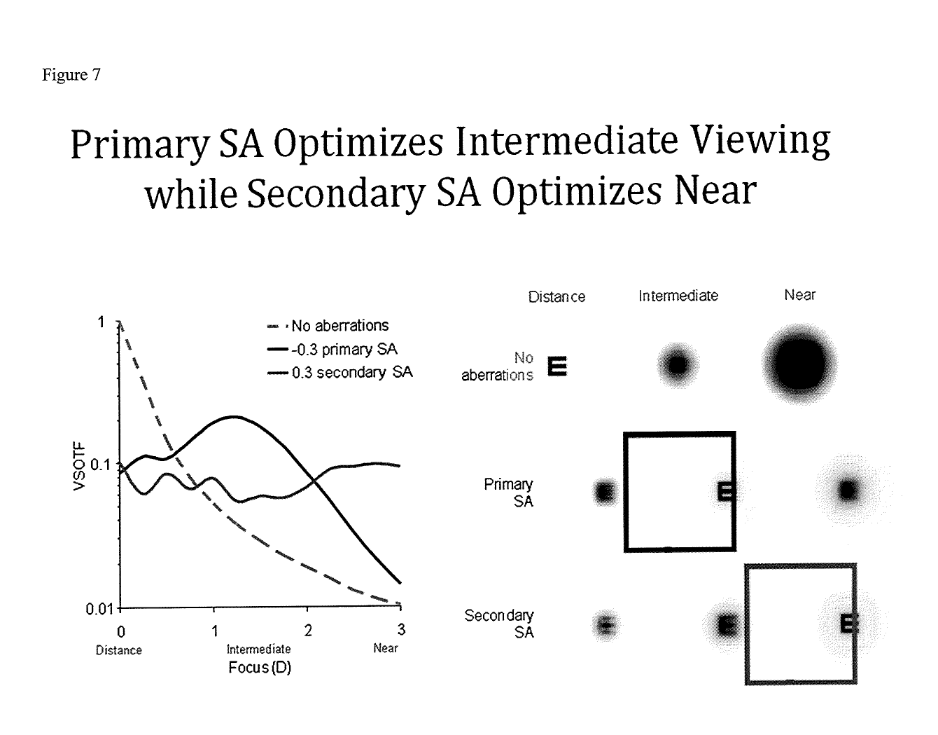

times focus. As shown in FIG. 7, primary SA optimizes intermediate viewing, while secondary SA optimizes near viewing. Also, primary SA can optimize intermediate or near depending on the magnitude of induce SA as discussed above. In the diffraction limited optical case, where no aberrations are present, as the VSOTF decreases as we move from distance to near on the graph, the convolved E on the right goes from clear to blurred beyond recognition.

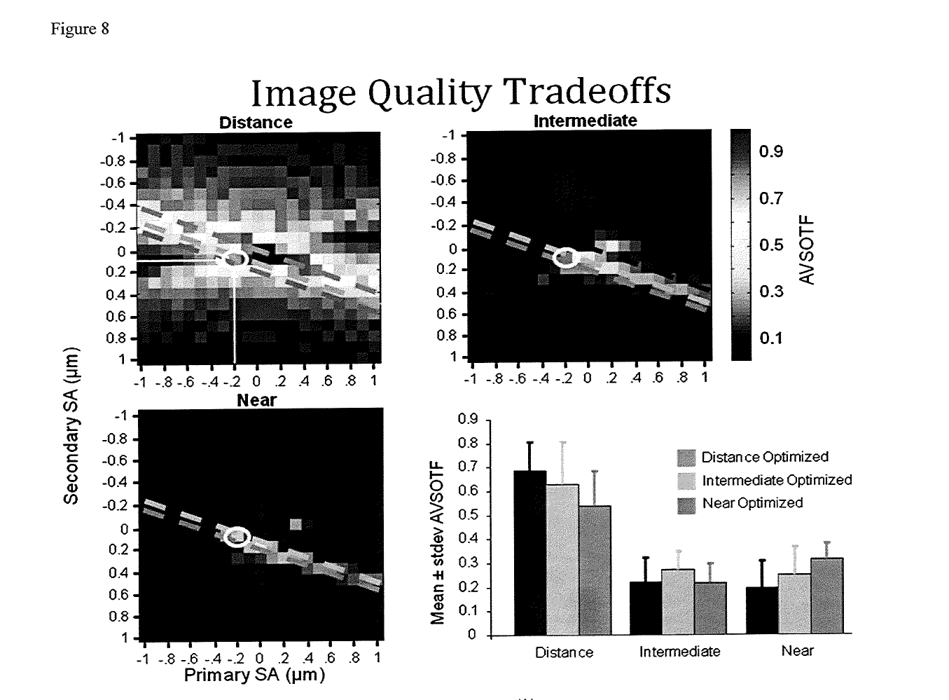

FIG. 8 shows image quality when combining different magnitude of primary and secondary SAs. Those color plots represent the area under the VSOTF curve for the distance, intermediate and near viewing zones, when primary SA, on the x-axis and secondary SA, on the y-axis interact through focus. Therefore, optimal multifocal designs are possible by using wavefront interactions between defocus and SAs to enhance through-focus image quality.

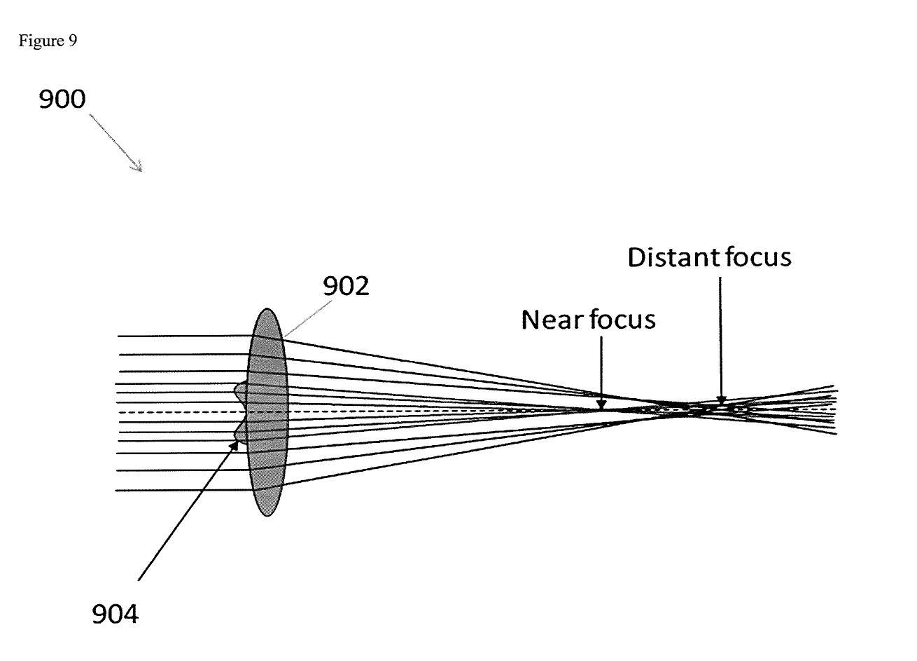

FIG. 9 shows an IOL 900 according to the preferred embodiment. As shown, the IOL includes a base lens 902 and a central optical subzone 904. The central optical subzone induces a higher-order aberration such as an SA.

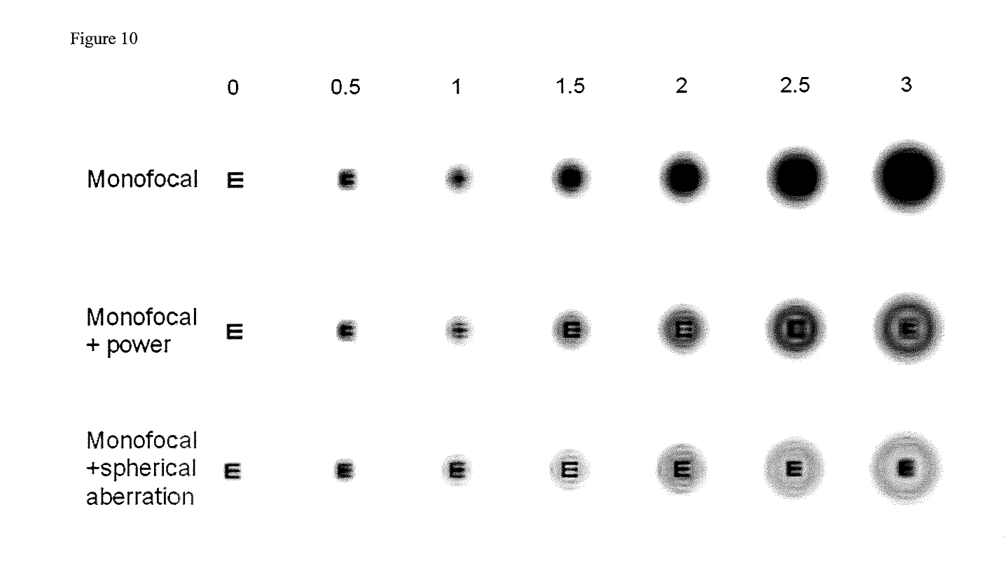

FIG. 10 shows the effectiveness of the invention. The top row shows the results of a monofocal lens; the middle row, monofocal plus central power like CrystalensHD; and the bottom row, monofocal plus central SA (the preferred embodiment). As can be seen, the preferred embodiment provides the best results.

The higher order aberration interaction concept can also work for a full optical zone diameter. FIGS. 4, 5, 6, 7 and 8 show that.

While a preferred embodiment has been set forth above, those skilled in the art who have reviewed the present disclosure will readily appreciate that other embodiments can be realized within the scope of the invention. For example, any higher-order aberration (spherical or higher) can be used. Therefore, the invention should be construed as limited only by the appended claims.

I claim:

1. A lens for correcting a patient's vision, the lens comprising:
a base lens; and
an optical subzone on the base lens, the optical subzone imparting a higher-order aberration.

2. The lens of claim 1, wherein the lens is an intraocular lens.

3. The lens of claim 1, wherein the optical subzone is centrally located.

4. The lens of claim 3, wherein the higher-order aberration comprises a spherical aberration.

5. The lens of claim 4, wherein the spherical aberration is a primary spherical aberration.

6. The lens of claim 4, wherein the spherical aberration is a higher order spherical aberration.

7. The lens of claim 6, wherein the higher order spherical aberration is a secondary spherical aberration.

8. The lens of claim 4, wherein the higher-order aberration comprises a combination of spherical aberrations.

9. A lens for correcting a patient's vision, the lens comprising:
an element for providing defocus; and
an element for providing a higher-order aberration that interacts with the defocus to correct the patient's vision.

10. The lens of claim 9, wherein the higher-order aberration comprises a spherical aberration.

11. The lens of claim 10, wherein the spherical aberration is a primary spherical aberration.

12. The lens of claim 10, wherein the spherical aberration is a higher-order spherical aberration.

13. The lens of claim 12, wherein the higher-order spherical aberration is a secondary spherical aberration.

14. The lens of claim 10, wherein the spherical aberration comprises a combination of spherical aberrations.

15. The lens of claim 9, wherein the elements define optical subzones.

16. The lens of claim 9, wherein the elements occupy a full optical zone.

17. A method for correcting vision in a patient's eye, the method comprising:
(a) correcting the vision using a base lens; and
(b) correcting the vision using an optical subzone on the base lens, the optical subzone imparting a higher-order aberration.

18. The method of claim 17, wherein steps (a) and (b) are performed by providing a lens.

19. The method of claim 18, wherein the lens is an intraocular lens.

20. The method of claim 17, wherein steps (a) and (b) are performed surgically.

21. The method of claim 17, wherein the optical subzone is centrally located.

22. The method of claim 21, wherein the higher-order aberration comprises a spherical aberration.

23. The method of claim 22, wherein the spherical aberration is a primary spherical aberration.

24. The method of claim 22, wherein the spherical aberration is a higher-order spherical aberration.

25. The method of claim 24, wherein the higher-order spherical aberration is a secondary spherical aberration.

26. The method of claim 22, wherein the spherical aberration comprises a combination of spherical aberrations.

27. A method for correcting a patient's vision, the method comprising:
(a) providing defocus in the patient's eye; and
(b) providing a higher-order aberration that interacts with the defocus to correct the patient's vision.

28. The method of claim 27, wherein the higher-order aberration comprises a spherical aberration.

29. The method of claim 28, wherein the spherical aberration is a primary spherical aberration.

30. The method of claim 28, wherein the spherical aberration is a higher-order spherical aberration.

31. The method of claim 30, wherein the higher-order spherical aberration is a secondary spherical aberration.

32. The method of claim 28, wherein the spherical aberration comprises a combination of spherical aberrations.

33. The method of claim 27, wherein steps (a) and (b) are performed in optical subzones.

34. The method of claim 27, wherein steps (a) and (b) are each performed in a full optical zone.

35. The method of claim 27, wherein steps (a) and (b) are performed by providing a lens.

36. The method of claim 35, wherein the lens is an intraocular lens.

37. The method of claim 27, wherein steps (a) and (b) are performed surgically.
